(19) United States
(12) Patent Application Publication
(10) Pub. No.: US 2024/0057871 A1
(43) Pub. Date: Feb. 22, 2024

(54) JUGULAR VENOUS PRESSURE MEASUREMENT DEVICES

(71) Applicant: JRAS Medical Inc., Saint John's (CA)

(72) Inventors: Andrew J. SMITH, Saint John's (CA); Jason D. ROBERTS, St. John's (CA); Andrew M.L. SMITH, St. John's (CA)

(21) Appl. No.: 18/133,683

(22) Filed: Apr. 12, 2023

Related U.S. Application Data
(63) Continuation of application No. 16/492,058, filed on Sep. 6, 2019, now abandoned.
(60) Provisional application No. 62/468,108, filed on Mar. 7, 2017.

(51) Int. Cl. A61B 5/00; A61B 5/021

(52) U.S. Cl. CPC A61B 5/0082; A61B 5/021; A61B 5/6844; A61B 2090/0807

(57) ABSTRACT

The present disclosure provides a device for measuring the jugular venous pressure of a patient. The device comprises a body defining a longitudinal enclosure and having a window along a length of the longitudinal enclosure to allow light to exit the longitudinal enclosure, a beam generator comprising a moveable portion mounted within the longitudinal enclosure, the beam generator configured to generate a sheet of light along a plane perpendicular to a longitudinal direction and at an adjustable position along the longitudinal direction, and direct the sheet of light out the window, an adjustment mechanism for adjusting the position of the moveable portion of the beam generator relative to the body along the longitudinal direction, and, a readout indicating the position of the sheet of light along the longitudinal direction.

### CROSS-REFERENCE TO RELATION APPLICATION

This application claims the benefit of priority of U.S. Provisional Patent Application No. 62/468,108, which was filed on Mar. 7, 2017 and is hereby incorporated herein by reference in its entirety.

### TECHNICAL FIELD

The present disclosure relates to devices for measuring the jugular venous pressure of a patient.

### BACKGROUND

Congestive heart failure (CHF) is a common and devastating health problem that affects upwards of 23 million individuals worldwide. Beyond incapacitating symptoms of shortness of breath and fatigue, long-term prognosis of CHF patients is extremely poor with only 50% and 10% of affected patients being alive at 5 and 10 years, respectively. Proper medical management of CHF is critical for improving symptoms and prolonging life and relies heavily on the physical examination. The primary goal of the physical exam among CHF patients is to evaluate for signs of volume overload, as excessive intravascular volume results in fluid backing up in the lungs causing shortness of breath and strain on the heart. Although there are multiple features that facilitate evaluation of volume status, the most informative is the jugular venous pressure (JVP). Assessment of the JVP involves attempting to visualize the height of a column of blood in a neck vein (internal jugular vein) just below the skin. Typically, the patient is placed in a semi-recumbent position, in the range of 30°-60° to the horizontal, with the head rotated away from the side being examined (10°-30° rotation). The clinician then examines the patient's neck to determine the height of the venous column demarked by the highest biphasic pulsation of the skin (as opposed to uniphasic pulsation of the adjacent carotid artery). Unfortunately, clinical assessment of the JVP is notoriously inaccurate and challenging to measure. This is a major clinical issue as optimal management of heart failure patients depends upon accurate assessment of the JVP. Inaccurate measurement may mislead clinical management decisions and result in adverse clinical outcomes.

Two major difficulties associated with measuring the JVP that can result in inaccurate measurements are: 1) Failing to correctly identify the height of the venous column of fluid along the neck, and 2) Ascertaining the height of the venous column relative to the sternal angle (a palpable landmark located along the chest at the level of the second ribs). The JVP is reported as height of the column of blood in the internal jugular vein, in centimeters, above the sternal angle with this value serving to guide subsequent medical therapy. An elevated JVP will generally trigger clinicians to diurese (remove fluid from) a patient in order to reduce volume overload, while a normal or low JVP reduces the likelihood that the patient is in active heart failure. A major challenge in ascertaining the correct height of the JVP relative to the sternal angle relates to the distance between the venous column in the neck and the sternal angle. Clinicians routinely make a visual estimation of the height, which is invariably error prone. More objective measurement of the JVP and standard training in medical school involves placing a ruler perpendicular to the horizontal plane and extending another straight edge from the ruler to the height of the venous column on the neck. This technique is cumbersome and difficult to perform. This is further compounded by clinicians rarely ever carrying two long rulers in their pocket during routine clinical rounds. As a result, this method is rarely ever performed in routine clinical practice.

Various devices have been proposed to facilitate measurement of the JVP, including (Patent US20100094141) and (Patent US20080294070). Neither of these techniques address the cumbersome features of the double ruler method, as both still involve extending a straight edge from a ruler aligned at the sternal angle.

The inventors have determined a need for improved devices for measuring the JVP.

### SUMMARY

One aspect provides a device for measuring jugular venous pressure of a patient. The device comprises a body defining a longitudinal enclosure and having a window along a length of the longitudinal enclosure to allow light to exit the longitudinal enclosure. A beam generator comprises a moveable portion mounted within the longitudinal enclosure. The beam generator is configured to generate a sheet of light along a plane perpendicular to a longitudinal direction and at an adjustable position along the longitudinal direction, and direct the sheet of light out the window. The device has an adjustment mechanism for adjusting the position of the moveable portion of the beam generator relative to the body along the longitudinal direction, and, a readout indicating the position of the sheet of light along the longitudinal direction. Some aspects also provide a level and/or a secondary light source integrated into the device.

Further aspects and details of example embodiments are set forth below.

### DRAWINGS

The following figures set forth embodiments in which like reference numerals denote like parts. Embodiments are illustrated by way of example and not by way of limitation in the accompanying figures.

FIG. 1 shows an example device for measuring JVP according to one embodiment of the present disclosure.

FIG. 2 is a longitudinal sectional view of the device of FIG. 1.

FIG. 2A shows a longitudinal sectional view of a portion of a device with a different beam generator and lens configuration according to another embodiment of the present disclosure.

FIG. 2B shows a longitudinal sectional view of a device for measuring JVP with an internal support rod according to another embodiment.

FIG. 2C shows a longitudinal sectional view of a portion of a device with the beam generator and lens configuration of FIG. 2A and the internal support rod of FIG. 2B.

FIG. 3 is a lateral sectional view of the device of FIG. 1.

FIG. 3A is a lateral sectional view of the device of FIG. 2B.

FIG. 4 shows the device of FIG. 1 projecting a light beam.

FIG. 5 shows a testing apparatus for the device of FIG. 1.

FIG. 5A shows the device of FIG. 1 with an adjusted scale applied thereto according to another embodiment of the present disclosure.

### DETAILED DESCRIPTION

The following describes an example embodiment of a device for measuring the JVP. The device has an elongated body which is oriented vertically when in use, and contains a beam generator that transmits a horizontal beam of light perpendicular to the vertical axis from an adjustable position along the body of the device. The horizontal beam of light passes through a lens to produce a sheet of light oriented along a substantially horizontal plane.

The vertical height of the horizontal sheet of light may be adjusted through adjustment of the height of a moveable portion of the beam generator within the device body. As discussed below, in some embodiments, the beam generator comprises a fixed light source and a moveable reflector, and in other embodiments the beam generator comprises a moveable light source. Also, in some embodiments the moveable portion of the beam generator comprises a lens, and in other embodiments a lens may be fixed and incorporated into a window on the device body.

The bottom edge of the device is designed to sit comfortably on the sternal angle of a patient inclined at a position approximately 45° (range: 30°-60°) from the vertical, with the device oriented vertically. The beam is then directed towards the side of the patient's neck (typically right) where the height of the jugular venous column can be visualized. The level of the horizontal sheet of light can then be adjusted to the height of the venous column by vertically adjusting the height of the moveable portion of the beam generator by means of an adjustment mechanism. When the beam is manually aligned with the height of the jugular venous column, the clinician simply reads the height (e.g. in cm) from a readout on the device. Manual vertical alignment may be assisted by detent stops or other tactile features. In some embodiments, the adjustment mechanism provides detent stops every 0.5 cm.

In the illustrated example, a button spirit level is provided at the top of the device body to enable the clinician to position the device vertically such that the beam is projected in a horizontal plane. In the illustrated example, the height of the horizontal sheet of light is adjusted using an adjustment mechanism in the form of a slider mechanism, and the readout comprises a scale next to the slider, as described further below. In other embodiments, the adjustment mechanism may comprise a different type of slider mechanism, a thumb wheel mechanism (e.g., a rack and pinion), a twisting or screw-type mechanism (e.g., twisting the base of the body to adjust the height of the sheet of light), another suitable mechanism.

The example device described below is ergonomically shaped and designed for use with either one or both hands. The device also includes a second light source in the form of a broad spectrum light emitting diode (LED) (e.g. a “white” LED) integrated into the bottom of the device body to serve as a pen-light for a variety of other clinical assessments. In other embodiments the device may also include a pocket clip which may incorporate a switch for the LED.

For simplicity and clarity of illustration, reference numerals may be repeated among the figures to indicate corresponding or analogous elements. Numerous details are set forth to provide an understanding of the examples described herein. The examples may be practiced without these details. In other instances, well-known methods, procedures, and components are not described in detail to avoid obscuring the examples described. The description is not to be considered as limited to the scope of the examples described herein.

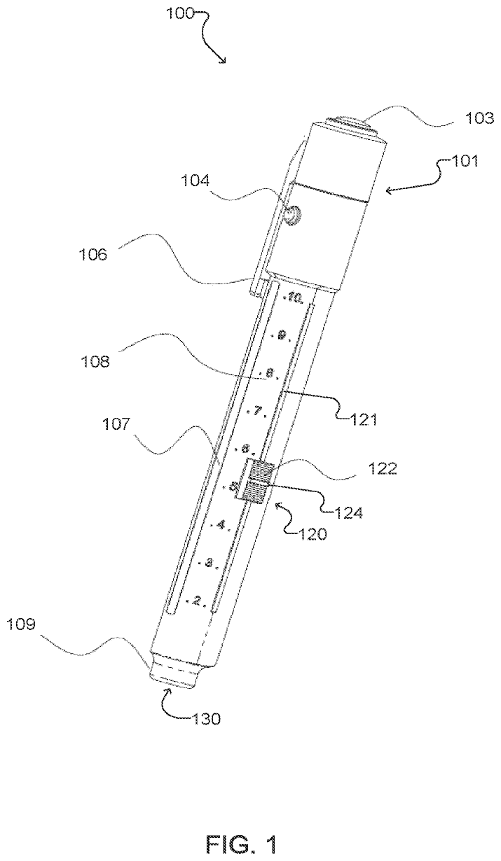

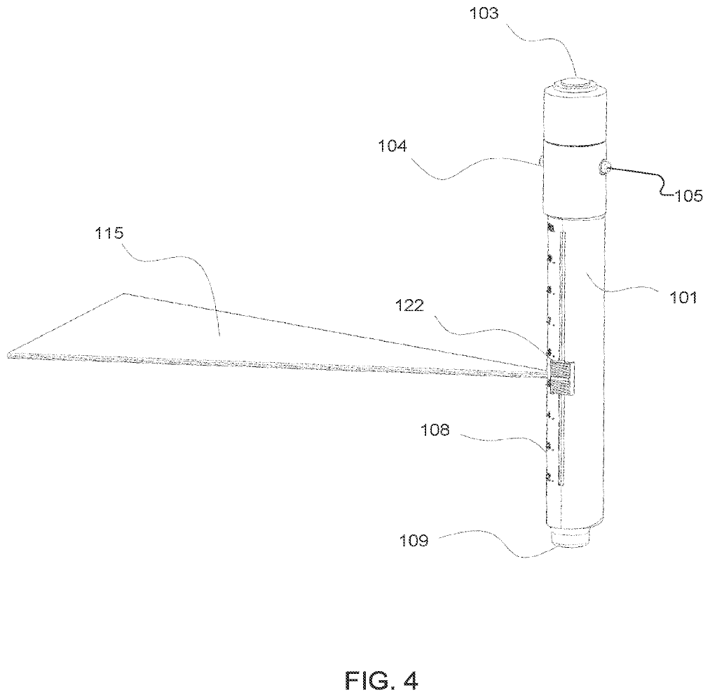

FIGS. 1, 2, 3 and 4 show an example device 100 for measuring JVP. The device 100 comprises an elongated body 101 that defines a longitudinal enclosure 102. The body 101 has a level 103 thereon for ensuring that the body 101 is vertical when measuring JVP as discussed below. In the illustrated example, the level 103 is on the top of the body 101. A beam switch 104, secondary light switch 105, and pocket clip 106 are also provided on an upper portion of the body 101 in the illustrated example. The beam switch 104 is operable to activate a beam generator 110 as discussed below. The secondary light switch 105 is operable to activate a secondary light (e.g. an LED) 130 at a bottom end 109 of the body 101. The switches 104 and 105 may, for example, comprise momentary switches or toggle on/off switches. The location and configuration of the switches 104 and 105 may differ in other embodiments. In some embodiments the beam switch 104 and/or the secondary light switch 105 may, for example, be incorporated into the button spirit level 103 or the pocket clip 106, into the slider 122, or into a lower portion of the body 101.

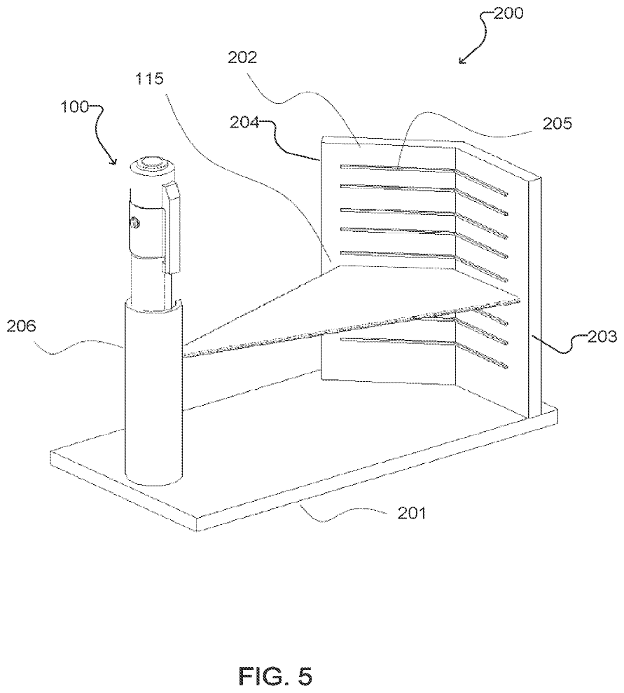

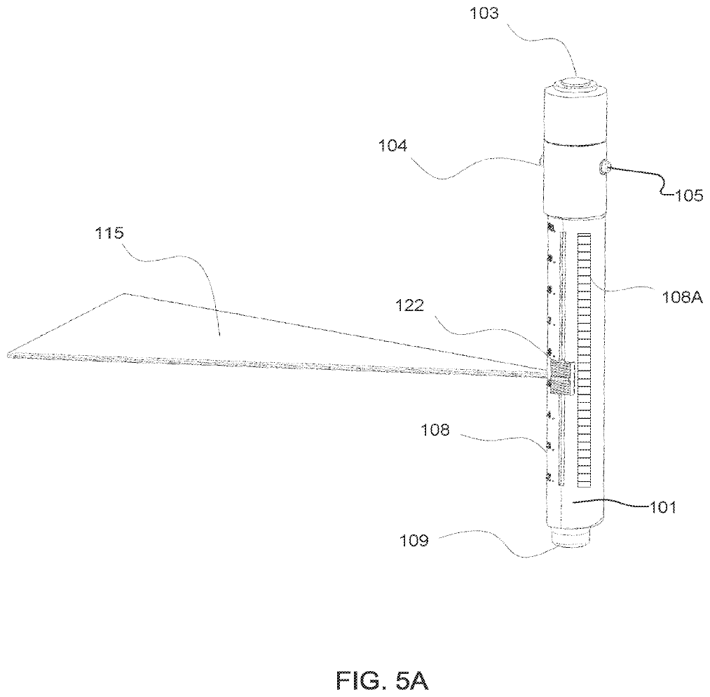

The beam generator 110 comprises a moveable portion adjustably mounted within the enclosure 102. The beam generator 110 is configured to generate a sheet of light 115 within a plane perpendicular to the longitudinal axis of device 100, as described further below, such that when device 100 is vertical, the sheet of light 115 is horizontal. The position of the moveable portion of the beam generator 110 within the enclosure can be adjusted by an adjustment mechanism 120. A window 107 is provided in the body along the length of the enclosure 102 to allow light to exit the body 101. A readout such as a scale 108 is provided on the body 101 for indicating the position of the beam generator 110 within the enclosure 102. In some embodiments, the scale 108 may be printed on the body after calibration of the device, or a corrected scale 108A may be adhered to the body 101, to compensate for any errors and accurately reflect the height of the sheet of light 115 at a distance of 15 cm away from the device 100, as discussed below with reference to FIGS. 5 and 5A.

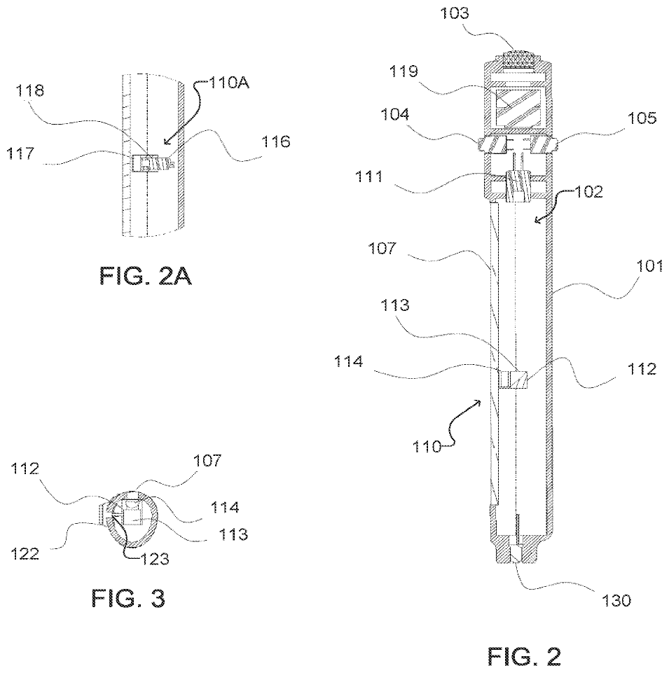

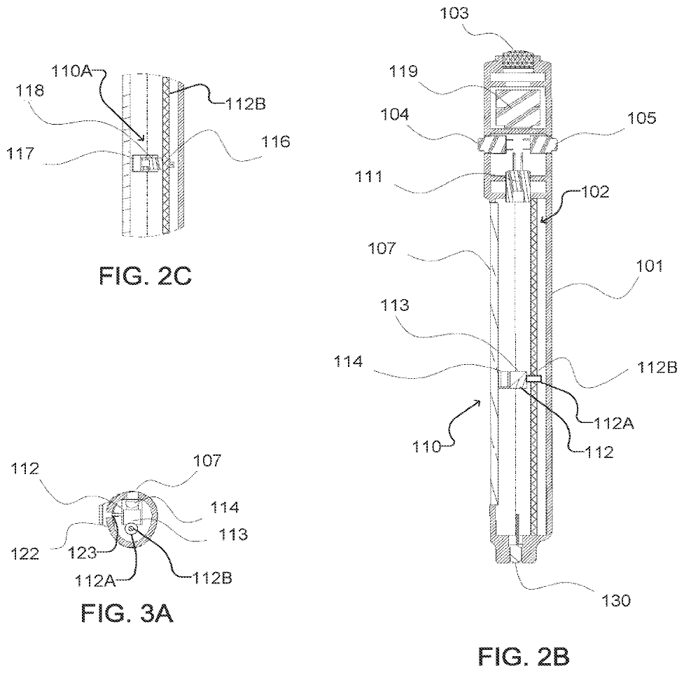

In the illustrated example, as best seen in FIG. 2 the beam generator 110 comprises a light source in the form of a laser 111 mounted in an upper portion of the body 101 above the enclosure 102. The moveable portion of the beam generator 110 comprises an optical assembly comprising a reflector 113 (e.g. a prism or mirror) and a lens 114, which are mounted on a platform 112 slidably mounted within the enclosure 102. A battery 119 is provided in the upper portion of the body 101 for powering the laser 111. In other embodiments, a laser or other light source could be mounted in a lower portion of the body 101 below the enclosure 102. In other embodiments, the lens 114 may be omitted, and the window 107 may comprise a lens to spread the light to generate the sheet 115. Other embodiments may have a beam generator 110A wherein the moveable portion comprises a laser or other light source 116 and lens 117 mounted on a slidable platform 118, as shown in FIG. 2A. In other embodiments, the moveable portion of the beam generator may comprise a light source mounted on a slidable platform with the window 107 functioning as a lens.

In the illustrated example, the adjustment mechanism 120 comprises a slider 122 connected to the platform 112 through a slot 121 in the body 101. The slot 121 is sealed with a flexible elastomer seal 123 configured to keep dust and contaminants out of the enclosure 102 while allowing movement of the slider 122. The slider 122 has an indicator mark 124 thereon adjacent to the scale 108. The slot 121 may have detent stops positioned periodically along its length, for example every 0.5 cm. The scale 108 and adjustment mechanism 120 are configured such that the indicator mark 124 is adjacent to a marking on the scale 108 indicating the height of the sheet of light 115 above the bottom end 109 of the body 101.

In some embodiments, the platform 112/118 is held in place by frictional bearing support from the edges of the body 101 around the slot 121. In other embodiments, one or more additional elements may provide support for the platform 112/118. For example, FIGS. 2B and 3A show an embodiment wherein a ring 112A attached to platform 112 slides along a supporting rod 112B extending longitudinally within the enclosure 102. The platform 112 could be coupled to the supporting rod 112B in other ways in other embodiments. For example, in some embodiments the platform 112 has an aperture therethrough sized to receive the supporting rod 112B such that the platform 112 can slide up and down the rod 112B. In some embodiments the platform 112 has a clip formed therein (e.g., a small ‘c’ integrated into its shape) and configured to engage the supporting rod 112B. As shown in FIG. 2C, the platform 118 of FIG. 2A could also be supported by a supporting rod 112B.

In operation, a clinician places the bottom 109 of the body 101 on a patient's sternal angle, and adjusts the position of the device to ensure the body 101 is vertical, as indicated by the level 103. The clinician then adjusts the height of the sheet of light 115 until it is aligned with the column of blood in the patient's vein, and reads the height from the scale 108.

FIG. 5 shows a testing apparatus 200 for testing the device 100. Apparatus 200 comprises a base 201, with a laser sight panel 202 comprising a perpendicular portion 203 and an angled portion 204 having gauge markings 205 thereon extending upwardly from the base 201. A sleeve 206 also extends upwardly from the base 201, and holds the device 100 perpendicularly to the base 201 such that the slider 122 is accessible and the scale 108 is visible. The perpendicular portion 203 and angled portion 204 are positioned at a predetermined distance to the sleeve 206 corresponding to a typical horizontal distance from the device to a patient's neck in a clinical setting (e.g. about 15 cm). A user can test the device 100 by inserting it on the sleeve 206 and activating the beam generator 110 to generate the sheet of light 115, then compare the height of the sheet of light 115 as measured by the gauge markings 204 with the height as indicated by the scale 108 on the device to ensure the heights match.

In some embodiments, the scale 108 may be printed on the body 101, or may be on a sticker or the like applied to the body 101, after calibration of the device 100 (for example by testing utilizing apparatus 200 or other testing apparatus) to account for any height mismatch. In some embodiments, a corrected scale 108A may be adhered to the body after testing, as shown in FIG. 5A.

The testing apparatus 200 is also useful for indicating any pitch or yaw angular errors in the orientation of the sheet of light 115. If the sheet of light 115 is not perpendicular to the device axis and ‘pitching’ up or down, this will result in a laser image line that is not parallel to the gauge markings 205 on the angled portion 204. Yaw angular errors are illustrated on the perpendicular portion 203 in a similar manner. If the sheet of light 115 is tipped (yaw) it will no longer be parallel on the surface of perpendicular portion 204 when compared to the markings 205. In some embodiments, the testing apparatus 200 also includes a mechanism for automatically activating the beam generator 110 when the device 100 is in the sleeve 206 (for example a physical feature attached to the sleeve 206 and positioned to contact the beam switch 104).

In some embodiments, the device 100 may be configured to interact with, or be incorporated into, other medical devices. For example, in some embodiments the device 100 includes a transducer or other type of sensor that generates a JVP signal based on the detected height, and a transmitter configured to send the JVP signal to another device such as an ultrasound or dialysis machine. In some embodiments, the device 100 transmits the detected height data to an ultrasound or dialysis machine via Bluetooth™ or other wireless transmission, or via wired transmission. In some embodiments, an ultrasound machine may be used to image the internal jugular vein (e.g. in long axis and/or transverse) and precisely determine the top of the column of fluid therein, which may be delineated on the patient's skin (either by the clinician visually identifying a feature on the skin at that height, or by applying a marking with, for example, a pen or marker). The device 100 may then be used as described above to determine the JVP height. In some embodiments, the device 100 may be incorporated into an ultrasound probe such that a single device can be used to image the internal jugular vein and determine the JVP height.

It will be appreciated that numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way, but rather as merely describing implementation of the various example embodiments described herein.

The description provides many example embodiments of the inventive subject matter. Although each embodiment represents a single combination of inventive elements, the inventive subject matter is considered to include all possible combinations of the disclosed elements. Thus if one embodiment comprises elements A, B. and C, and a second embodiment comprises elements B and D, then the inventive subject matter is also considered to include other remaining combinations of A, B, C, or D, even if not explicitly disclosed.

Although the embodiments have been described in detail, it should be understood that various changes, substitutions and alterations can be made herein. Moreover, the scope of the present application is not intended to be limited to the particular embodiments of the process, machine, manufacture, composition of matter, means, methods and steps described in the specification.

The present disclosure may be embodied in other specific forms without departing from its spirit or essential characteristics. The described embodiments are to be considered in all respects only as illustrative and not restrictive.

1. A device for measuring jugular venous pressure of a patient comprising:
a body defining a longitudinal enclosure and having a window along a length of the longitudinal enclosure to allow light to exit the longitudinal enclosure;
a beam generator comprising a moveable portion mounted within the longitudinal enclosure, the beam generator configured to direct light out the window to generate a sheet of light along a plane perpendicular to a longitudinal direction and at an adjustable position along the longitudinal direction;
an adjustment mechanism for adjusting the position of the moveable portion of the beam generator relative to the body along the longitudinal direction; and,
a readout indicating the position of the sheet of light along the longitudinal direction.

2. The device of claim 1 wherein the beam generator comprises a laser mounted at one end of the longitudinal enclosure, and the moveable portion of the beam generator comprises a reflector for redirecting the laser's light slidably mounted within the longitudinal enclosure.

3. The device of claim 2 wherein the moveable portion of the beam generator comprises a lens for generating the sheet of light slidably mounted with the reflector within the longitudinal enclosure.

4. The device of claim 2 wherein the window comprises a lens for generating the sheet of light.

5. The device of claim 1 wherein the moveable portion of the beam generator comprises a laser slidably mounted within the longitudinal enclosure.

6. The device of claim 5 wherein the moveable portion of the beam generator comprises a lens for generating the sheet of light slidably mounted with the laser within the longitudinal enclosure.

7. The device of claim 5 wherein the window comprises a lens for generating the sheet of light.

8. The device of claim 1 wherein the adjustment mechanism comprises a slider mechanism having a slider connected to one or more components of the beam generator and extending out a longitudinal slot in the body.

9. The device of claim 8 wherein a plurality of detent stops are positioned periodically along the longitudinal slot.

10. The device of claim 8 comprising a flexible elastomer seal along the longitudinal slot configured to keep dust and contaminants out of the enclosure while allowing movement of the slider.

11. The device of claim 8 herein the readout comprises a scale on the body adjacent to the longitudinal slot and an indicator mark on the slider.

12. The device of claim 11 wherein the scale is printed on the body after calibration of the device.

13. The device of claim 11 wherein the scale is printed on a sticker attached to the body after calibration of the device.

14. The device of claim 1 comprising a light emitting diode integrated into a base of the body.

15. The device of claim 1 wherein the moveable portion is frictionally supported by walls of the body.

16. The device of claim 1 wherein the moveable portion is frictionally supported by a supporting rod extending within the longitudinal enclosure.

17. The device of claim 1 comprising a level on the body for indicating when the longitudinal direction is vertically aligned.

18. A testing apparatus comprising:
a base for resting on a horizontal surface;
a sleeve extending upwardly from the base and configured to support a device according to claim 1 in a vertical orientation;
a panel extending upwardly from the base having horizontal gauge markings thereon, the panel comprising a perpendicular portion oriented perpendicularly to a line extending from the device in the sleeve, and an angled portion oriented at an acute angle to a line extending from the device in the sleeve, whereby a sheet of light generated from the device is parallel to the gauge markings on both the perpendicular portion and the angled portion of the panel when the device is functioning correctly.

19. The testing apparatus of claim 18 wherein the gauge markings include height indicators.

20. The testing apparatus of claim 18 wherein the sleeve comprises a mechanism for automatically activating a beam generator of the device.
